(19) United States
(12) Patent Application Publication
(10) Pub. No.: US 2006/0069356 A1
(43) Pub. Date: Mar. 30, 2006

(54) Plunger for a syringe and syringe

(76) Inventor: Norbert Witowski, Wolfenbuttel (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/210,109

(22) Filed: Aug. 24, 2005

Related U.S. Application Data
(60) Provisional application No. 60/603,633, filed on Aug. 24, 2004.

(30) Foreign Application Priority Data
Aug. 24, 2004 (DE) 10 2004 040 969.2

(51) Int. Cl. A61M 5/315

(52) U.S. Cl. 604/222; 604/230

(57) ABSTRACT

The invention relates to a plunger for a syringe that has a barrel. The plunger has a core element (8) with a peripheral surface (20). The plunger has a ring element (11) with an outer peripheral surface (21) that surrounds the core element (8) at a distance. The ring element (11) is coated on its outer peripheral surface (21) with a lubricating material (22). The core element (8) and the ring element (11) are connected to one another by arms (19).

This application claims the benefit of the filing date of U.S. Provisional Application Ser. No. 60/603,633 filed Aug. 24, 2004, which is incorporated by reference herein.

This invention relates to a plunger/plug for a syringe and to a syringe into which such a plunger is inserted.

A plunger for a syringe and a syringe into which such a plunger is inserted are known from, for example, WO 98/17339 A1, which is incorporated herein by reference.

It is a general problem in syringes that the force of friction between the plunger and the inside wall of the cylinder of a syringe is high. Therefore, conventionally at least the inside cylinder wall of the syringe was coated with silicone oil. It is uncertain, however, whether in the application of the contents of the syringe to a patient the silicone oil is applied at the same time. Therefore, in the indicated WO 98/17339 A1, it is proposed that the silicone oil be omitted and the friction reduced by a suitable material of the plunger. It is proposed that the plunger be made from polytetrafluoroethylene (PTFE). PTFE is known, i.a., under the trade name “Teflon.” PTFE has the disadvantage, however, that it cannot be formed by injection molding. It must be turned on a lathe from a “solid.” Since PTFE is relatively expensive, this leads to high costs in the production of the plunger. Furthermore, the scrap in turning from a solid piece is high. For PTFE, it must be disposed of as special waste; this likewise increases costs.

In German Patent DE 3346 351 published on Apr. 9, 1992, a plunger is described that is provided at least partially with a Teflon coating. Such a plunger makes contact with the inside cylinder wall with beads that exert a specific pressure on the inside cylinder wall, by which a sealing effect is to be ensured. The beads are made such that the perpendicular line of the bead surface pointing away from the plunger points toward the wall of the barrel. The patent uses Teflon to form a protective surface between the medium and the rubber part of the plunger. In this case, it is solely an insulating function; the outstanding sliding properties of Teflon were not further considered here. In this publication, the concept of siliconization was not mentioned. Thus, it can be assumed that in this case, one skilled in the art reads this text to understand that it is a syringe that otherwise corresponds to general technical knowledge (e.g., DIN Standard 13 098, part 1, item 4.4) and that it is likewise siliconized on the inside of the cylinder. Autoclaving is not mentioned.

In DE GM 19 73 042 dated Nov. 23, 1967, a plastic syringe made of artificial resin is described that consists of a plastic cylinder with a needle opening and a plunger opening, a plunger and a piston. The syringe as the important feature has a plunger made in the shape of a shell or cup and consists of commercially available material. Such a sealing lip, however, can only be made in combination with a lubricant, especially with silicone oil. If silicone is not present, the sealing lips would change due to high friction and they would turn down, yielding laterally to the force. In this publication, the concept of siliconization is not mentioned either. Thus, it can also be assumed here that one skilled in the art reads this text to understand that it is a syringe that otherwise corresponds to general technical knowledge (e.g., DIN Standard 13 098 part 1, item 4.4) and that is likewise siliconized on the inside of the cylinder. Autoclaving is not mentioned.

In Patent AT-E 68 979, a filled, terminally sterilized syringe is described. The syringe consists of plastic. The syringe has a cylinder with a distal end with a syringe outlet piece. The syringe outlet piece is sealed by a closure. Before filling, the inside wall of the syringe is coated with silicone oil. The syringe is sealed after filling with a flexible rubber plunger that can also slide in the cylinder due to the silicone oil.

The process for producing a filled, terminally sterilized syringe begins with removing the scrap particles or other impurities from the closure and the plunger. Microbial impurities on the closure and the plunger are destroyed. The cylinder is washed with a plurality of water jets in order to remove pyrogens and scrap particles. Then, silicone oil is applied to the inside wall of the syringe. The closure is thereupon slipped onto the syringe outlet piece. The syringe is filled with the contrast agent through its proximal end. The syringe is then sealed with the plunger. This assembled and filled syringe is sterilized in an autoclave. In doing so, in addition to ordinary autoclave pressure, an additional support pressure is produced in the autoclave.

From the publication by Venten and Hoppert (E. VENTEN and J. HOPPERT (1978) Pharm. Ind. Vol. 40, No. 6, pages 665 to 671), prefilled, terminally sterilized, single-dose disposable syringes that are provided with a silicone oil layer on the inside wall are known. The single-dose disposable syringes that have a plunger on the proximal end are filled distally through the roll edge. The roll edge is then sealed by a gasket, a flange cap fixing the gasket on the roll edge. (M. JUNGA (1973) Pharm. Ind. Vol. 35, No. 11a, pages 824 to 829). The prefilled, single-dose disposable syringes are then transferred to an autoclave. This autoclave can be controlled with respect to temperature and pressure.

European Patent Application EP 0553 926 (date of application: Jan. 26, 1993) describes a process for terminal sterilization of a prefilled plastic syringe or glass syringe, the syringe containing a contrast agent. The inside wall of the disposable syringes is coated with silicone oil. The syringe consists of a barrel that has a syringe outlet piece on the distal end. In addition, single-dose disposable syringes are cited in the form already described previously in Venten and Hoppert. The disposable syringes have an open proximal end that can be closed by a plunger that can slide in the disposable syringe. The plunger is connected to a piston.

WO 95/12482 describes a process for producing prefilled plastic syringes that are filled with a contrast agent. The inside wall of the syringe is coated with silicone oil. The syringes consist of a cylinder and a syringe outlet piece on the distal end that is prepared for a needle mount. Furthermore, the syringe comprises a plunger that can slide in the cylinder. It seals the proximal end of the syringe. The syringe has been produced according to a process that leads to pyrogen-free objects. Likewise, there are no longer any particles. The syringe is filled through the proximal end; here, the syringe outlet piece is sealed with a closure. The filled syringe is sealed with the plunger.

After the syringe parts come from the casting mold, they are blown off with gas to remove particles. The syringe is then washed and provided with a lubricant. Afterwards the syringe is sterilized so that the syringe can be further treated, stored or transported, as desired.

The disadvantage in the known syringes is that silicone oil must be used to reduce the friction between the plunger and the inside wall of the syringe. As advantageous as a rubber plunger is with respect to elastic forces, equally problematic is the behavior with respect to the sliding friction. Adhesive friction is even more unfavorable. Specifically for long-term storage of the syringe with the plunger inserted, the adhesive friction plays a very important role. Furthermore, for rubber plungers, the cold flow behavior must also be watched. Since the latter is a quantity that cannot be ignored, rubber plungers with considerable pretensioning must be used. This is even more important if the filled syringes with the rubber plunger inserted are then autoclaved. The cold flow behavior of the rubber plunger is a function of the temperature. High pretensioning in this case is necessary when the plunger is inserted. Here, the friction in all cases is so high that without silicone oil, handling of the syringes is not possible.

Even if rubber sealing lips are used, in the static state they do not adequately seal or they suffer from cold flow behavior, especially in autoclaving. In this case, considerable pretensioning must also be used so that silicone oil is also essential in this case.

A solid plunger made of Teflon has a major disadvantage in syringes that are exposed to a thermal load. Here, temperature fluctuations from −10° C. to +40° C. are enough to allow the plastics of the syringe wall to expand relative to the very hard Teflon material. Autoclaving is especially a load on the plunger or the syringe in that one of the two exhibits cold flow behavior that after cooling of the syringe results in leaks. In order to effectively seal the plunger, due to the low elastic behavior of the solid Teflon plunger, high pretensioning is necessary, which thus results in high friction. In plastic syringes, such a Teflon plunger cannot be used since the cold flow behavior of the plastic syringe wall increases the inside diameter of the syringe at the level of the plunger during autoclaving. In this way, gaps form through which liquid can escape uncontrolled from the syringe. For this reason, air can also be sucked in in an uncontrolled manner. Glass syringes that do not have such cold flow behavior must still be coated with silicone oil on the inside wall to reduce friction that is caused by the very high pretensioning. When such a syringe is autoclaved, the Teflon plunger yields relative to the harder glass due to the cold flow behavior. Here also, after cooling, spaces and gaps that allow the syringe to become leaky are the result.

Thus, the object of the invention is to offer a prefilled, sterile, medical syringe in which the addition of a lubricant in the form of, for example, silicone oil is eliminated, without adversely affecting the sliding capacity of the plunger that should consist of plastic in the syringe, and, moreover, adequate sealing of the contents of the syringe by the plunger relative to the outside area of the syringe is ensured.

This object is achieved by a plunger according to claim 1.

Preferred embodiments of the plunger are indicated in subclaims 2 to 16.

The object is also achieved by a syringe according to claim 17.

One advantageous embodiment of the syringe is indicated in claim 18.

The syringe according to the invention can be easily filled with contrast agent and terminally sterilized. Preferred examples of the contrast agent are amidotrizoic acid, gadopentetic acid, gadobunol, gadolinium, iopamidol, iopromide, iotrolan, and iotroxic acid.

The plunger according to the invention has a core element. A ring element surrounds the core element at a distance. The two elements are connected by arms.

This procedure has the advantage that the plunger is not made solid. Conventional materials such as thermoplastic, from which the plunger can be produced, have a high coefficient of thermal expansion. If, therefore, a syringe with a solid plunger of thermoplastic is terminally sterilized in an autoclave at 121°, the thermoplastic plunger due to the high coefficient of thermal expansion is deformed so greatly that it is destroyed.

Furthermore, the ring element according to the invention is coated on its outer peripheral surface with a lubricating material. This lubricating material makes it possible for the plunger to be able to move easily in the cylinder of a syringe; this would not be possible if the peripheral surface of the plunger were to consist of thermoplastic, since thermoplastic has a high coefficient of friction with materials for barrels such as glass or plastic. Since the peripheral surface of the ring element is coated with an expensive lubricating material, the cost can be kept low.

Since the core element and the ring element are separate from one another and there is a distance between the two, there is less solid material for the core element and the ring element. The radial extension of the material is less, less than the radius. For the same coefficient of expansion, therefore, the thermal expansion becomes less due to the reduced length. Thus, even when the syringe with the plunger according to the invention is heated, the plunger is not destroyed by the thermal expansion.

The stiff core into which the plunger rod can be screwed ensures that the plunger rod in an injection does not deform the plunger and increases the friction between the plunger and a cylinder wall.

When the arms extend radially from the core element to the ring element, the arms can accommodate the thermal expansion by compressing.

When the arms are attached tangentially to the core element, they do not run radially from the inside to the outside. When the core element and the ring element undergo thermal expansion, the arms are bent at their attachment points. Therefore, compression of the arms is not necessary at all.

When the ring element is made in one piece, the injection mold can be easily produced.

When the ring element is divided into a plurality of ring segments that are spaced apart in the peripheral direction, the ring segments can deform tangentially during thermal expansion instead of radial deformation that could lead to destruction, so that destruction does not occur.

If the ring element has radially peripheral projections and especially projections with an undercut, the lubricating material adheres better to the outer peripheral surface of the ring material.

When the lubricating material has a sealing lip, it is possible to achieve especially good sealing. In particular, it is possible to achieve dynamic sealing that occurs when the plunger is moved toward the distal end of the syringe. This is especially advantageous for a lubricating material with a low coefficient of friction between the lubricating material and the cylinder wall of the syringe.

When the core element, the arms and the ring element are to be made in one piece from the same material, an especially economical injection molding process for this integral component is possible.

Preferred materials for the core element, the arms and the ring element are thermoplastic, polypropylene or polycarbonate.

Preferred materials for the sliding element are thermoplastic elastomers. Preferred examples are: Evoprene 968, Cawiton PR 6173 B, Cawiton PR 6173 D, Cawiton PR 6173 E, Cawiton PR 6173 F, Thermoflex 55 1A5+900, Uniprene 7010 RS 70+801 (SEBS), Santoprene 181-57 W180 (TPV).

The syringes are conventionally rotationally symmetrical, only the finger holding devices and equipment holding devices and occasionally also the syringe outlet piece deviate from the symmetry. The syringe outlet piece can thus be arranged eccentrically. A Luer-Lock is especially preferred since it takes effect only when contrast agents are applied if mechanical pump devices are used. Even in manual application, the Luer-Lock and the tube that is connected to it prevent unintentional movements of the physician from being directly transferred to the needle. Furthermore a simple Luer nozzle and also a record nozzle are known.

It is also possible to weld and thus seal the syringe outlet piece. It is then advantageous for the syringe outlet piece to have scoring that easily allows opening of the syringe outlet piece before use.

The proximal and the distal end of the syringe must be sealable. The distal end is sealed by a closure that can be placed on the syringe outlet piece. The syringe outlet piece in this industrial property right encompasses the cover of the barrel. Furthermore, the syringe outlet piece encompasses a tube that leads to the needle or the hose, an end piece that is in contact with the needle or the hose, and a cylinder with a thread on the inside, the cylinder surrounding the end piece and bearing a thread for a Luer-Lock, for example. Here, the syringe outlet piece can be one piece or several pieces. The cover can be rounded, flat or pyramidal. Mixed forms are also conceivable. The plunger seals the proximal end of the syringe. It must be able to slide in the cylinder and must reliably withhold the medium from the environment. It should be as little permeable to gases and liquids as possible. Temperature fluctuations must also be accommodated without malfunction. Conventionally, the plunger is not provided with its own piston when the syringe is mechanically emptied. Rather, the piston that is part of the pump device fits into a closure within the plunger so that movement of the plunger is easily possible (cf. EP 0 584 531).

The terms proximal and distal are defined from the viewpoint of the treating physician. On the distal end is the syringe outlet piece to which, for example, the needle or a hose that leads to the needle is connected. On the proximal end is the plunger that presses the medium through the distal end during application. Movement of the plunger can take place manually or else mechanically. The expression plunger also comprises plungers. For manual actuation of the syringe, it is helpful for the operator if the syringe on the proximal end has finger holding devices. Here, the finger holding devices conventionally have at least one surface as an abutment for the index finger and the middle finger, the surface of the finger holding device being essentially perpendicular to the axis of the barrel. Different models are known for mechanical pump devices. A syringe then bears preferably one or more equipment holding devices on preferably the proximal end. Such a mechanical pump is described especially well in EP 0 584 531 (Reilly at al. Date of application Jul. 21, 1993). Mixed forms of the finger holding device and equipment holding device are also possible.

The medium in the filled syringe is a mixture of a fluid medium and at least one gas. In this case, the gas volume should be as small as possible; a gas volume that assumes a value of zero is desirable. The medium can be a liquid, a solution, a suspension or an emulsion. These appearance forms are described in W. SCHRÖTER et al., (1987) Chemistry; Facts and Laws, 14th Edition, Leipzig, on pages 23 ff.

A fluid medium that is a contrast agent is preferred. In this connection, there are the following contrast agents with the generic names: amidotrizoic acid, gadopentetic acid, gadobutrol, gadolinium EOB-DTPA, iopamidol, iopromide, iotrolan, and iotroxic acid.

Medical syringes according to the invention are advantageous, the plunger being made complementary to the shape of the distal end of the syringe in order to minimize the residual volume that cannot be removed from the syringe in spite of completely depressing the plunger.

A medical syringe according to the invention is preferred, the plunger having convex, plane or concave execution or else pyramidal, conical, truncated pyramidal, truncated conical or hemispherical configurations, depending on the design of the end of the syringe the protuberance pointing distally or proximally.

A syringe according to the invention in which the syringe can be autoclaved is preferred.

A syringe according to the invention in which the syringe can be autoclaved at a support pressure is more preferred.

It is a good idea that at least the syringe body be cast or injected in a sterile room at at least 250° C.

A syringe according to the invention is advantageous in which the sterile syringe can be packaged in a sterile container that has at least one gas-permeable, but not germ-permeable wall.

Foreign bodies must be removed from the syringe. Foreign bodies are all the particles that are not made of the material of the syringe and the medium and the detached fragments of the syringe.

Pyrogens are substances that as fragments of bacteria provoke a human immune response. They are especially lipopolysaccharides, i.e., cell wall components of gram-negative bacteria.

After the syringe has been partially assembled, it is possible if necessary to again remove foreign bodies from the syringe. Foreign bodies are all the particles that are not made of the material of the syringe and the medium and the detached fragments of the syringe.

Radiation sterilization and chemical sterilization processes are especially suited as sterilization processes.

Chemical sterilization processes include treatment with ethylene oxide, propan-3-olide and diethyl dicarbonate; furthermore, hydrogen peroxide and an ozone/steam mixture are known.

Sterilization with high-energy radiation is also possible. Here, gamma rays and x-rays are known.

Optionally, the parts of the syringe are packed sterile in bacteria-tight, but gas-permeable films or aluminum. Sterilization takes place using thermal and/or chemical sterilization, with gamma rays or x-rays, neutron rays or beta rays or a mixture of the aforementioned rays. Treatment with hydrogen peroxide or an ozone/steam mixture is preferred.

Then, the syringe body is filled by the distal or proximal end, either the plunger or the closure sealing the opposite end. Then, the fill opening is sealed by the closure or the plunger.

The distal end is sealed with a closure or by welding of the distal end. In the welding, the distal end has scoring proximally to the welding. In this way, the distal end can be easily opened after welding.

In the next step, the syringe or cartridge is thermally sterilized in an autoclave or sterilizer with hot air or by means of microwaves.

So that the plunger does not travel within the cylinder, it is advantageous if the plunger is fixed during sterilization.

Optionally, it is possible to build up a support pressure in the sterilization space of the autoclave or the sterile chamber by a gas in the sterilization space, the pressure on the outside surface of the syringe being greater than, equal to or less than the pressure on the inside surface of the syringe. The support pressure can be defined as the pressure that corresponds to the sum of the partial pressures in the sterilization space minus the partial pressure of the steam.

It is advantageous if the plunger is re-adjusted after sterilization. This ensures that the plunger is in the optimum position. Occasionally, the friction between the plunger and cylinder is so great that adjustment of the plunger into the stable position in which there is no pressure difference between the inside and the outside of the syringe does not take place automatically.

At this point, optical checking is advantageous. This ensures that particles that are located in the syringe are detected. Syringes with particles should be discarded in this case.

Packaging of the sterilized syringe in a container and sterilization of the filled container are especially important. This process can take place in a sterile room. This step is especially advantageous because only in this way is there the safety of offering a syringe that is also externally sterile to the attending physician. In this way, the danger of contamination can be reduced. For the syringes that are to be mechanically emptied, this advantage also applies since the physician also touches the syringe here. Often the syringes that are to be mechanically emptied are used in sterile operating rooms. Only sterile or disinfected materials may be brought into these rooms. Thus, a syringe that is to be mechanically emptied must also be externally absolutely sterile.

It is furthermore advantageous that the filled and terminally filled syringe is packaged in sterile plastic film and/or aluminum foil under optionally aseptic conditions. In this case, it is advantageous that the syringe be packaged in as sterile a blister as possible, if necessary aseptic conditions prevailing.

Then, the syringe that is in the container is externally re-sterilized by treating the syringe with ethylene oxide, propan-3-olide and/or diethyl dicarbonate. Furthermore, hydrogen peroxide and an ozone/steam mixture are known.

The packaging of the sterilized syringe in a container and the sterilization of the filled container can take place in a sterile room. This step is especially advantageous because only in this way is there the safety of offering a syringe that is also externally sterile to the attending physician. In this way, the danger of contamination can be reduced. This advantage also applies to syringes that are to be mechanically emptied, since the physician also touches the syringe here. Often, the syringes that are to be mechanically emptied are used in sterile operating rooms. Only sterile or disinfected materials may be brought into these rooms. Thus, a syringe that is to be mechanically emptied must also be externally absolutely sterile.

Chemical sterilization processes include treatment with ethylene oxide, propan-3-olide and diethyl dicarbonate, Furthermore, hydrogen peroxide and an ozone/steam mixture are known. These processes are described in:
G. SPICHER (1990) Möglichkeiten und Grenzen der Sterilisation mit Gasen und ionisierenden Strahlen im Vergleich mit den klassischen Sterilisationsverfahren [Possibilities and Limits of Sterilization with Gases and Ionizing Rays Compared to Classical Sterilization Processes], Pharma Technologie, Vol. 11, No. 4, pages 50-56;
H. HÖRATH (1990) Rechtliche Rahmenbedingungen der Sterilisation mit Ethylenoxid und Formaldehyd [Legal Outline Conditions of Sterilization with Ethylene Oxide and Formaldehyde], Pharma Technologie, Vol. 11, No. 4, pages 57-64;
J. SCHUSTER (1990) Die Praxis der betrieblichen Ethylenoxid-Sterilisation und Versuche zu ihrer Optimierung [Practice of Operational Ethylene Oxide Sterilization and Tests on its Optimization], Pharma Technologie, Vol. 11, No. 4, pages 65-71;
M. MARCZINOWSKI (1990) Praktische Durchführung der Formaldehyd-Sterilisation [Practical Execution of Formaldehyde Sterilization], Pharma Technologie, Vol. 11, No. 4, pages 72-76.

Sterilization with high-energy radiation is likewise possible. Here, gamma rays and x-rays are known. Likewise, neutron rays, beta rays and alpha rays are used.

The invention likewise encompasses a process for producing a prefilled, sterile syringe that comprises the following features:
a) Sterile production of the syringe parts or cleaning and sterilization of the produced syringe parts,
b) Assembly of the parts,
   (i) In this case, the distal end is sealed in proximal filling or
   (ii) The proximal end is sealed by the plunger in distal filling,
c) Optionally sterilization of the assembled syringe,
d) Proximal or distal filling, depending on the still remaining opening,
e) Proximal sealing by the plunger or distal sealing by an outlet closure or welding of the outlet,
f) Sterilization of the filled and sealed syringe,
g) Optionally sterile packaging of the syringe in a container with at least one surface that is gas-permeable, but not germ-permeable.

Such processes are described in detail in EP 0 227 401, EP 0 553 926 and U.S. Pat. No. 5,207,983. Reference is likewise made to the bibliographic reference of E. Venten and J. Hoppert, Pharm. Ind. Vol. 40, No. 6, (1978). In this publication, including the literature cited therein, sterilization methods are considered in detail. The Venten and Hoppert publication is part of the application by citation. It is especially advantageous if a combination consists of the syringes according to the invention and an application device composed of an injectomat and of connections, the injectomat being a pump system and the connections joining the outlet of the syringe to the patient. Such an injectomat is described in, e.g., EP 0 192 786.

The invention furthermore comprises a combination of a prefilled, terminally sterilized syringe according to the invention, and an application device consisting of an injectomat and connections, the injectomat being a pump system and the connections joining the outlet of the syringe to the patient. Such a pump system is described in the publication EP 0 584 531.

Other features and functionalities of the invention follow from the description of embodiments below using the figures. Of the figures:

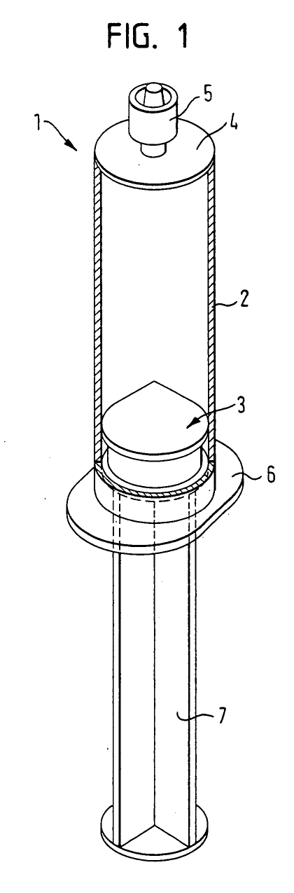

FIG. 1 shows a syringe as it is used for the invention;

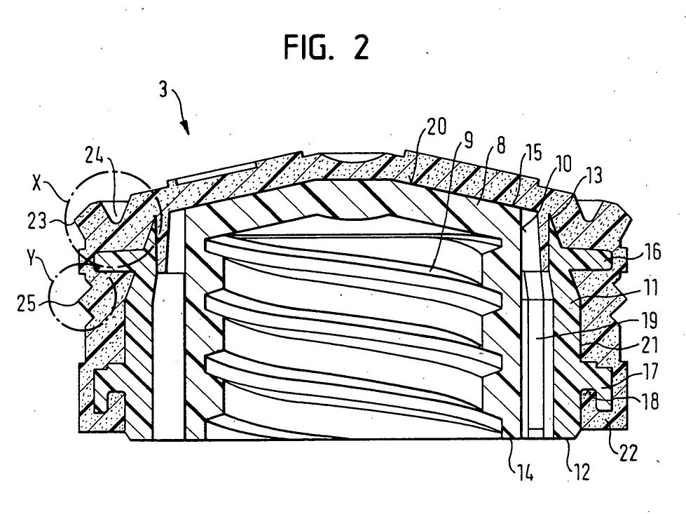

FIG. 2 shows a cross-sectional view of a plunger as it is used for the invention;

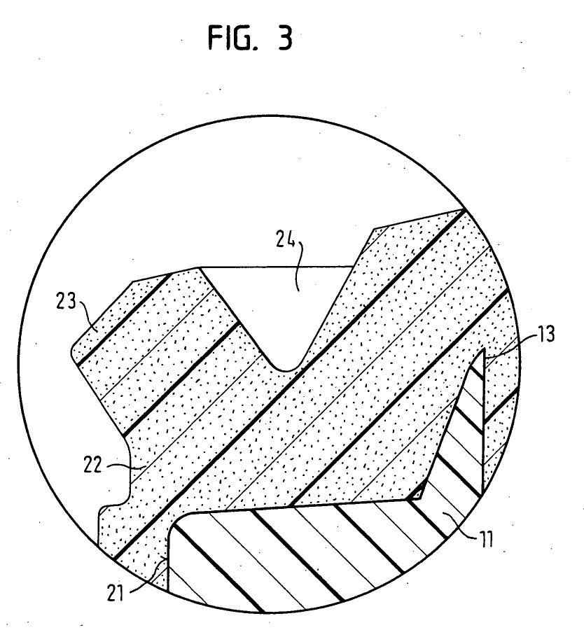

FIG. 3 shows an enlargement of section X in FIG. 2;

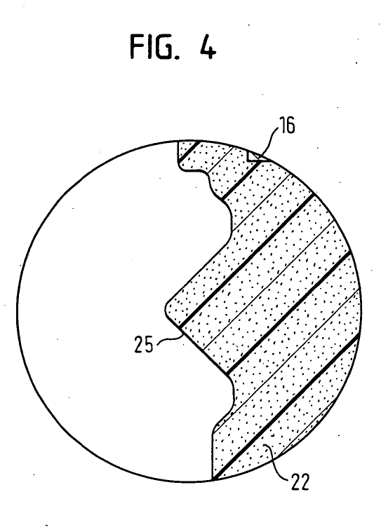

FIG. 4 shows an enlargement of section Y in FIG. 2;

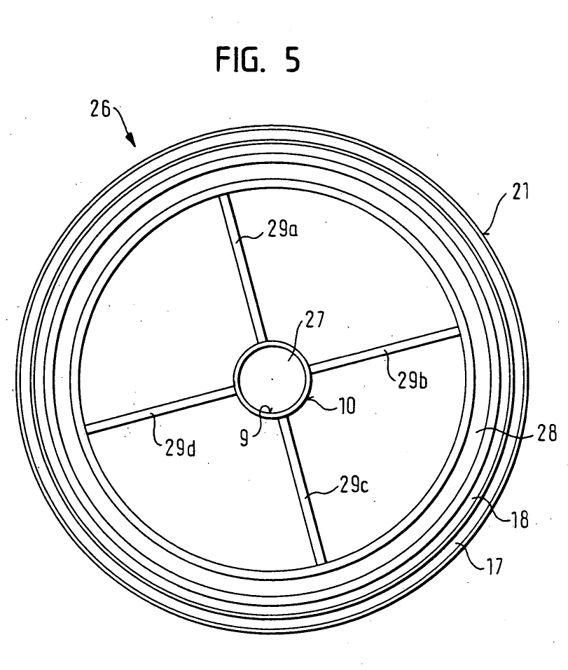

FIG. 5 shows a view of a plunger of a first embodiment viewed from the distal side of the syringe;

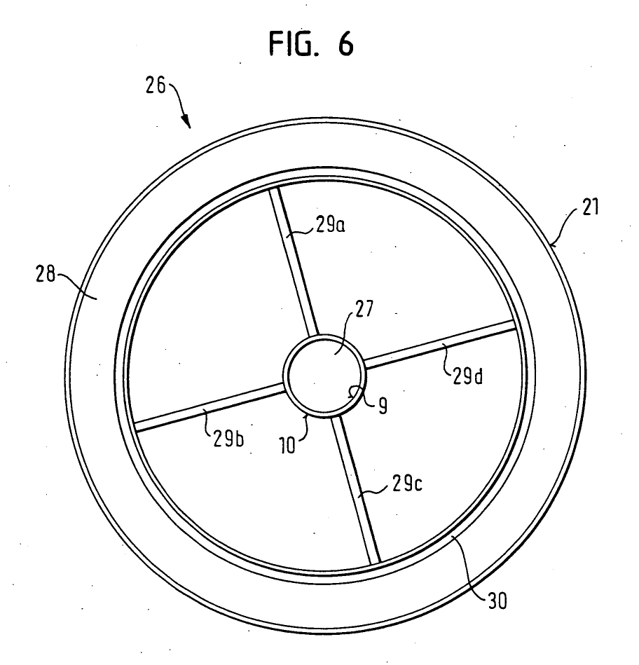

FIG. 6 shows a view of the plunger of the first embodiment viewed from the proximal side;

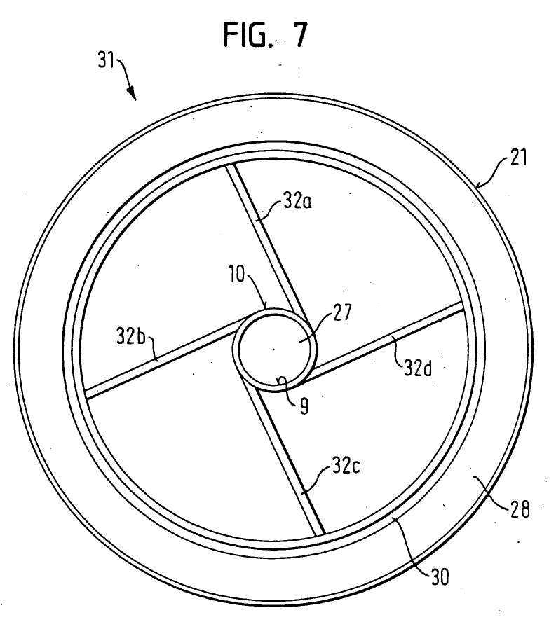

FIG. 7 shows a view of a plunger of a second embodiment viewed from the proximal side;

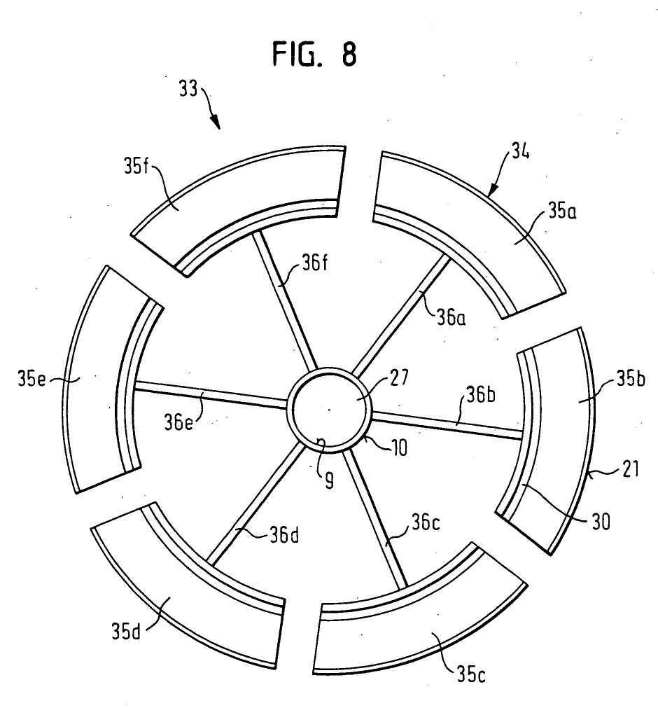

FIG. 8 shows a view of a plunger of a third embodiment viewed from the proximal side;

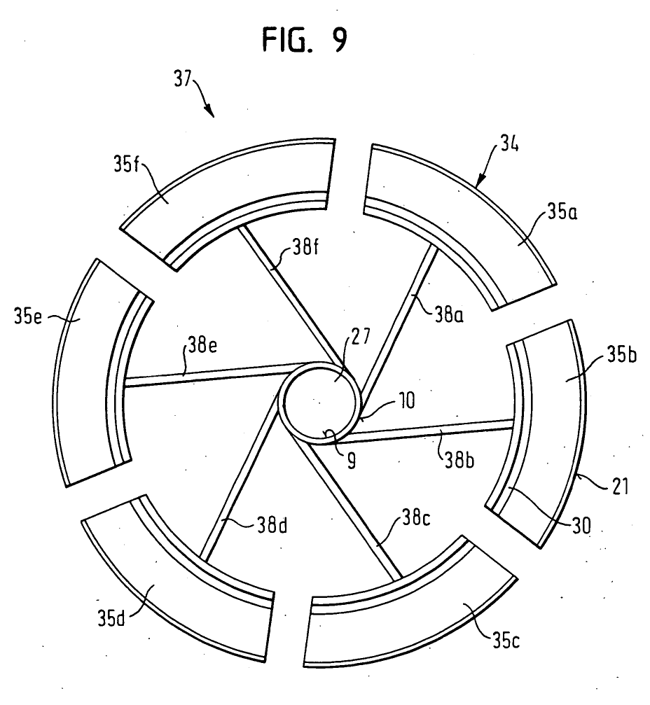

FIG. 9 shows a view of a plunger of a fourth embodiment viewed from the proximal side;

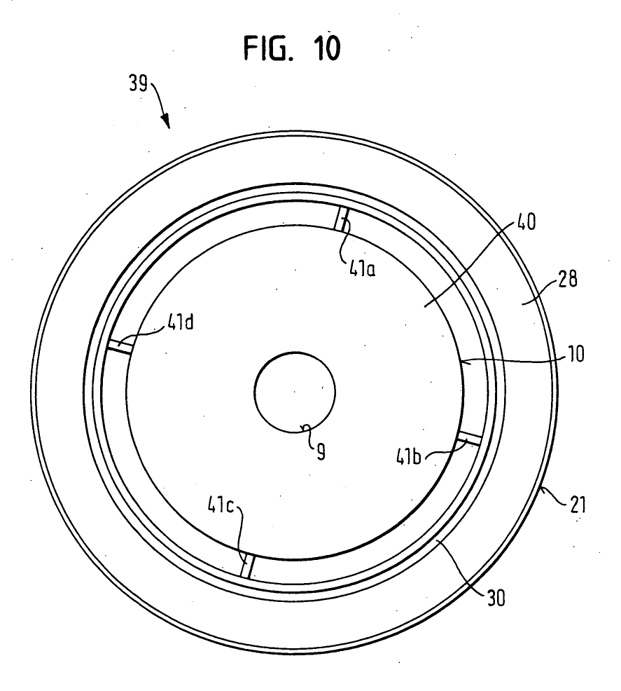

FIG. 10 shows a view of a plunger of a fifth embodiment viewed from the proximal side;

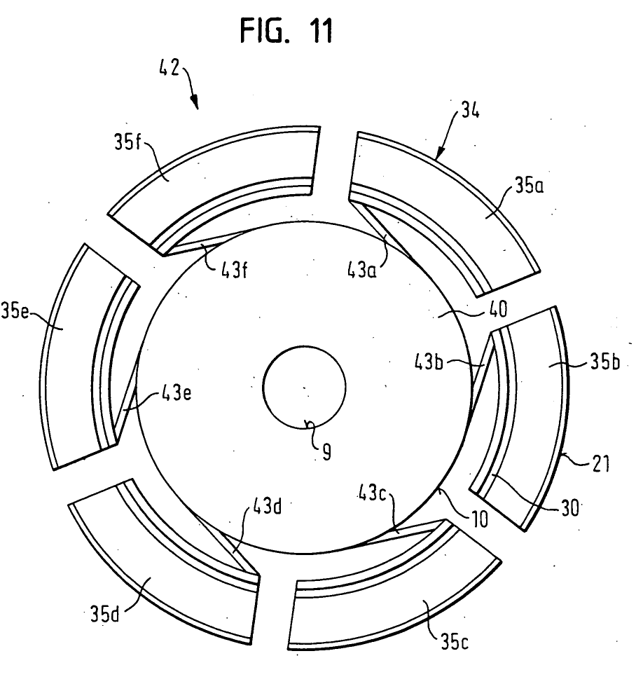

FIG. 11 shows a view of a plunger of a sixth embodiment viewed from the proximal side;

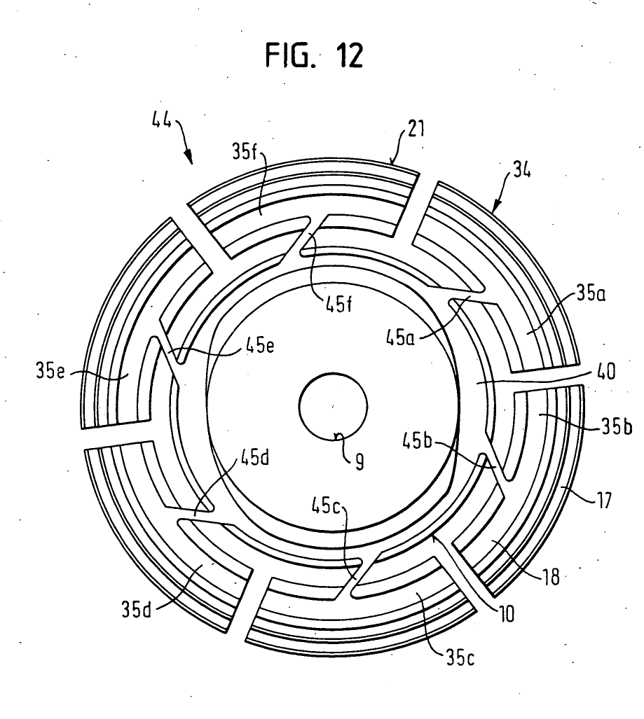

FIG. 12 shows a view of a plunger of a seventh embodiment viewed from the distal side; and

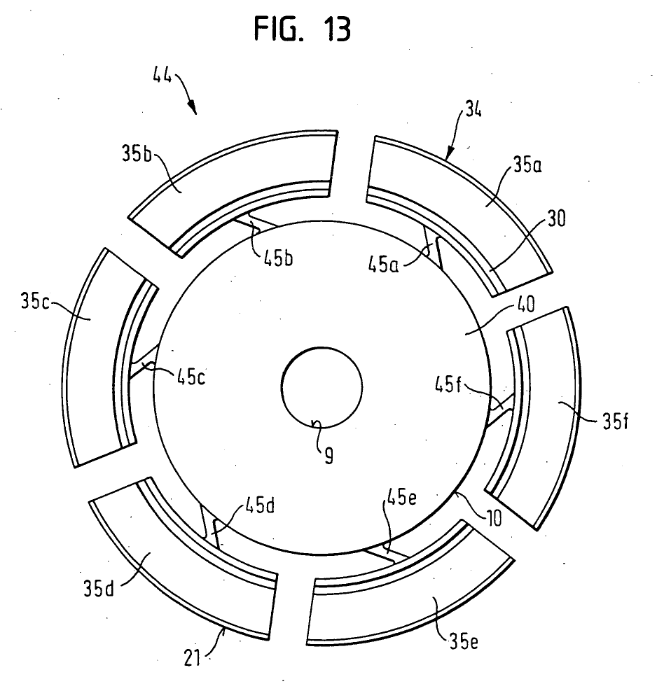

FIG. 13 shows a view of a plunger of the seventh embodiment viewed from the proximal side.

FIG. 1 shows a syringe as it is used for this invention.

As is shown in FIG. 1, a syringe 1 has a cylinder 2 and a plunger 3. The cylinder 1 on the distal end has a cap 4 with a central opening and a nozzle 5 for holding a needle or a hose. On one proximal end of the cylinder 2 is a flange 6 for holding or insertion into a motorized actuating element (not shown). From the proximal side, a plunger rod 7 is screwed into the plunger 3. Reference is made to E. Venten and J. Hoppert, 1978, Pharm. Int. Vol. 40, No. 6, pp. 665 to 671 for a more detailed explanation, the contents of this publication being incorporated herein by reference.

The terms “top,” “bottom,” and “lateral” used below relate to the position of the syringe 1 as is shown in FIG. 1. This means that “top” relates to the direction to the distal end, and “bottom” relates to the direction to the proximal end.

The general structure of the plunger 3 is now described with reference to FIG. 2.

The plunger 3 has a generally cup-shaped core element 8 that is open to the bottom. On the inner peripheral surface of the core element 8, there is an internal thread 9 into which the plunger rod 7 can be screwed. The core element 8 has a lateral peripheral surface 10.

The lateral peripheral surface 10 of the core element 8 is surrounded at a distance by an annular ring element 11. The ring element 11 extends vertically, i.e., from a proximal end 12 to a distal end 13 to essentially the same height as the core element 8 extends from a proximal end 14 to a distal end 15. The ring element 11 on its top distal section has a peripheral, radially extending projection or flange 16. The ring element 11 on its bottom proximal section has a peripheral projection 17 with an undercut 18.

The core element 8 and the ring element 11 are connected by a plurality of arms 19. The arms extend essentially over the height of the ring element 11 or of the core element 8. According to one modification, however, the arms 19 extend over a smaller height than the core element 8 and the ring element 11.

The core element 8, the ring element 11 and the arms 19 are produced integrally from thermoplastic by injection molding. Especially polypropylene is suited as thermoplastic.

A layer 22 of a lubricating material is applied to a top outer surface 20 of the core element 8 and to the lateral outer peripheral surface 21 of the ring element 11. According to one modification, the layer 22 of lubricating material is applied only to the lateral peripheral surface 21 of the ring element 11.

The layer 22 of lubricating material on the top section has a peripheral sealing lip 23. In FIG. 2, a circle that surrounds the sealing lip 23 is labelled X. The circle X is shown enlarged in FIG. 3. As can be seen in FIG. 3, the sealing lip 23 has a cross section that extends essentially obliquely to the top and outward, the upper tip of the rectangle being bevelled. The sealing lip 23 together with the top of the layer 22 made of lubricating material forms a V-shaped peripheral depression 24. The sealing lip 23 is made relatively long and has a relatively flat angle relative to the axis of the plunger 3. In this way, two functions are performed. The dynamic behavior is such that when the syringe 1 is actuated, i.e., when the plunger 3 is pushed to the distal end, the sealing lip is pressed against the inside wall of the cylinder. In the static condition, i.e., without motion, the sealing lip 23 is pressed by its elasticity against the inside wall of the cylinder and safeguards sealing.

Furthermore, at roughly half the height of the outer peripheral surface of the layer 22 of lubricating material, there is a peripheral, additional sealing lip 25 that projects to the outside. In FIG. 2 a circle that surrounds the sealing lip 25 is labelled Y. The circle Y is shown enlarged in FIG. 4. The sealing lip 25 forms an additional safety when the plunger 3 is sealed against the cylinder 2 of the syringe.

As shown in FIG. 2, the layer 22 made of lubricating material extends into the undercut 18. This and providing a peripheral projection 16 ensure good holding of the layer 22 made of lubricating material on the ring element 11. The layer 22 made of lubricating material is sprayed after injection molding of the core element 8, the arms 19 and the ring element 11 onto the still warm formed piece consisting of the core element 8 and the ring element 11. In this way, an especially intimate connection of the layer 22 of lubricating material and the core element 8 and the ring element 11 is additionally produced.

The layer 22 is formed from a thermoplastic elastomer. This results in that a smaller coefficient of friction of μ between the layer 22 of lubricating material and the inside wall of the cylinder 2 of the syringe 1 can be achieved.

Embodiments of the arrangement of the core element 8, the arms 19 and the ring element 11 are explained below with reference to FIGS. 5 to 13.

FIG. 5 shows the plunger of a first embodiment from the bottom, before the layer 22 of lubricating material is applied, and FIG. 6 shows the plunger of the first embodiment from the top, before the layer 22 of lubricating material is applied. The plunger 26 has a core element 27 and a ring element 28 that are connected by four arms 29a, 29b, 29c, and 29d. The core element 27 is relatively small compared to the entire plunger 26; its diameter is roughly ⅛ of the diameter of the plunger. On the top (see FIG. 6), a groove 30 is shown in which the layer 22 made of lubricating material enters when sprayed so that the layer 22 made of lubricating material is reliably held on the top side of the plunger 26. The arms 29a to 29d extend radially from the core element 27 to the ring element 28. When the plunger of the first embodiment is heated, the arms 29a to 29d are compressed in their lengthwise direction and thus accommodate thermal expansion. In the first embodiment shown in FIGS. 5 and 6, four arms 29a to 29d are provided. According to one modification, however, there can be three or five or more arms. On the peripheral surface, the layer 22 on the lubricating material is held by the flange 16 and the projection 17.

FIG. 7 shows a plunger 31 of a second embodiment from the top. The plunger 31 of the second embodiment is formed essentially identically to the plunger 26 of the first embodiment. Therefore, a description of the same features is not repeated. The plunger 31 of the second embodiment, however, differs from the plunger 16 of the first embodiment in that the arms 32a, 32b, 32c, and 32d do not extend radially from the core element 27 to the ring element 28, but rather the arms 32a to 32d are attached tangentially to the core element 27. This procedure has the advantage that when the core element 27 and the ring element 28 expand when heated, the arms 32a to 32d are not compressed, but rather exert a rotary motion on the core 27. Therefore, the thermal expansion of the plunger 31 when heated can be absorbed better.

FIG. 8 shows a plunger 33 of the third embodiment from the top. The plunger 33 of the third embodiment corresponds essentially to the plunger 26 of the first embodiment. Therefore, the description of the same features is not repeated. The plunger 33 of the third embodiment, however, differs from the plunger 26 of the first embodiment in that a ring element 34 is not made through-going, but rather is divided into ring segments 35a, 35b, 35c, 35d, 35e and 35f that are spaced apart from one another in the peripheral direction. The arms 36a, 36b, 36c, 36d, 36e and 36f extend radially from the core element 27 to the ring segments 35a to 35f. The third embodiment has the advantage that when the ring element 34 undergoes thermal expansion, the gaps between the ring segments 35a to 35f can accommodate the expansion. The force on the plunger 33 can be better accommodated during thermal expansion. In the third embodiment shown in FIG. 8, there are six ring segments 35a to 35f and six arms 36a to 36f. According to one modification, however, there can be three to five or seven or more ring segments and arms.

FIG. 9 shows a plunger 37 of a fourth embodiment. The plunger 37 of the fourth embodiment corresponds essentially to the plunger 33 of the third embodiment. Therefore, the description of the same features is not repeated. The plunger 37 of the fourth embodiment, however, differs from the plunger 33 of the third embodiment in that the arms 38a, 38b, 38c, 38d, 38e and 38f do not act radially on the core element 37, but rather the arms 38a to 38f act tangentially on the core element 27 as in the plunger 31 of the second embodiment. Thus, the plunger 37 of the fourth embodiment combines the advantages that the second embodiment and the third embodiment have. Thermal expansion of the core element 27 and of the ring element 34 can be extremely well absorbed.

FIG. 10 shows a plunger 39 of a fifth embodiment from the top. The plunger 39 corresponds essentially to the plunger 26 of the first embodiment. Therefore, a description of the same features is not repeated. The plunger 39 of the fifth embodiment, however, differs from the plunger 26 of the first embodiment in that a core element 40 is made larger compared to the entire plunger 39. The diameter of the core element 40 of the fifth embodiment corresponds roughly to ⅔ of the diameter of the plunger 39 of the fifth embodiment. This enables high stability for the core element 40. The arms 41a, 41b, 41c and 41d can be made very short. This embodiment is especially advantageous when major temperature increases are not expected so that the thermal expansion remains limited.

According to one modification of the fifth embodiment, the arms 41a to 41d are not radially attached to the core element 40, but they are provided tangentially, as in the second embodiment. Thus, in the modification, the same advantage can be achieved as in the second embodiment.

FIG. 11 shows a plunger 42 of a sixth embodiment. The plunger 42 of the sixth embodiment corresponds to the plunger 39 of the fifth embodiment. Therefore, the description of the same features is not repeated. The plunger 42 of the sixth embodiment, however, has the ring element 34 that is divided into ring segments 35a to 35f, as in the plunger 37 of the fourth embodiment. In the plunger 42 of the sixth embodiment, the arms 43a, 43b, 43c, 43d, 43e and 43f are attached tangentially to the ring element 40. In contrast to the plunger 37 of the fourth embodiment, however, the arms 43a to 43f are not attached in the middle of the ring segments 35a to 35f, but rather the arms 43a to 43f are each attached to one end of the ring segments 35a to 35f, as is shown in FIG. 11. The procedure for the plunger 42 of the sixth embodiment therefore additionally has the advantage that the ring segments 35a to 35f can fold to the inside on their free ends. Thus, the ring segments 35a to 35f during thermal expansion can better accommodate thermal expansion.

According to one modification of the sixth embodiment, the ring segments 35a to 35f are not uniformly thick, as is shown in FIG. 11, but rather the ring segments 35a to 35f are beveled on their free ends on the inside. In this way, the ring segments 35a to 35f can be turned better to the inside, the arms 43a to 43f being used as pivots. Thus, the ring segments 35a to 35f can better yield thermal expansion.

FIGS. 12 and 13 show a plunger 44 of a seventh embodiment. The plunger 44 of the seventh embodiment corresponds to the plunger 42 of the sixth embodiment. Therefore, the description of the same features is not repeated. The plunger 44 of the seventh embodiment, however, differs from the plunger 42 of the sixth embodiment in that the arms 45a, 45b, 45c, 45d, 45e and 45f connect the core element 40 to the ring segments 35a to 35e that are not radially aligned, but that rather are somewhat sloped relative to the radial direction. The arms 45a to 45f are each attached in the middle of the ring segments 35a to 35f. The arms 45a to 45f are not sloped so greatly that they are tangentially attached to the core element 40, but they are located somewhat nearer to the radial direction. With the plunger 44 of the seventh embodiment, thermal expansion can also be absorbed when the plunger and the syringe are heated.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2004 040 969.2, filed Aug. 24, 2004 and U.S. Provisional Application Ser. No. 60/603,633, filed Aug. 24, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Plunger (3, 26, 31, 33, 37, 39, 42, 44) for a syringe (1) with a barrel (2), with:
a core element (8, 27, 40) with an outer peripheral surface (10), and
a ring element (11, 28, 34) that surrounds the core element at a distance, with an external peripheral surface (21),
the ring element being coated with a lubricating material (22) on its outer peripheral surface and
the core element and the ring element being connected by arms (19, 29, 32, 36, 38, 41, 43, 45).

2. Plunger according to claim 1, in which the arms (29, 36, 41) extend radially from the core element (27, 40) to the ring element (28, 34).

3. Plunger according to claim 1, in which the arms (32, 38, 43) extend tangentially from the peripheral surface (20) of the core element (27, 40) to the ring element (28, 34).

4. Plunger according to claim 1, in which the ring element (28) is made in one piece.

5. Plunger according to claim 1, in which the ring element (34) has a plurality of ring segments (35a, 35b, 35c, 35d, 35e, 35f) that are spaced apart in the peripheral direction.

6. Plunger according to claim 1,
with a bottom that faces a proximal end of the syringe (1), and
with a top that faces a distal end of the syringe (1).

7. Plunger according to claim 6, in which on the outer surface (21) of the ring element (11), a projection (23) that extends radially around the outer surface (21) of the ring element (11) on a section that faces the top is provided.

8. Plunger according to claim 6, in which on the outer surface (21) of the ring element (11), a projection (17) that extends radially around the outer surface (21) of the ring element (11), with an undercut (18) on the section of the outer peripheral surface (21) of the ring element (11), which section faces the bottom, is provided.

9. Plunger according to claim 6, in which the core element (8) is open to the bottom and has an inside thread (9) for holding a plunger rod (7).

10. Plunger according to claim 6, in which the lubricating material (22) is formed on the top of the core element (8) and of the ring element (11).

11. Plunger according to claim 8, in which the lubricating material (22) extends into the undercut (18).

12. Plunger according to claim 6, in which the lubricating material (22) has an annular sealing lip (23) on the section facing the top, the sealing lip (23) adjoining an inside wall of the barrel (2) to form a seal.

13. Plunger according to claim 1, in which the core element (8), the arms (19) and the ring element (11) are formed in one piece from the same material.

14. Plunger according to claim 13, in which the material is a thermoplastic, preferably polypropylene.

15. Plunger according to claim 1, in which the lubricating material (22) is a thermoplastic elastomer, preferably Santoprene.

16. Syringe (1) with a barrel (2) made of glass or plastic and a plunger (3) that is inserted into the barrel (2) as it is claimed in claim 1.

17. Syringe according to claim 16 that is prefilled.
